(19) United States
(12) Patent Application Publication
(10) Pub. No.: US 2004/0200861 A1
(43) Pub. Date: Oct. 14, 2004

(54) Plunger damping means

(75) Inventors: Neil Barry Ward, Hamilton (NZ); Peter John Tasker, Auckland (NZ)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: SIMCRO TECH LIMITED

(21) Appl. No.: 10/807,403

(22) Filed: Mar. 24, 2004

(30) Foreign Application Priority Data
Mar. 24, 2003 (NZ) 524938

(51) Int. Cl. A61M 5/00; G01F 11/06

(52) U.S. Cl. 222/321.6

(57) ABSTRACT

A dispensing apparatus has a plunger reciprocable in a barrel, the plunger head defining with the barrel a variable volume pump chamber, a way-one fluid inlet valve for allowing fluid to flow into the chamber on increase of the chamber volume and a fluid outlet valve allowing fluid to flow out of the chamber when the volume decreases, a spring biasing the plunger toward a first position and a plunger actuator for actuating the plunger toward a second position, and a plunger damper for decelerating the plunger when moving toward its first position but before it reaches the end of its first position.

### TECHNICAL FIELD

The present invention relates to dispensing apparatus which use plungers to dispense fluid, and in particular, but not exclusively, to applicators for treating animals.

### BACKGROUND ART

Many dispensing devices of the prior art use a plunger which is reciprocable within a barrel to deliver fluid via a nozzle to a desired location, which, if the apparatus is a medical applicator, may be the mouth or back of an animal.

A problem with the dispensers of the prior art, in particular those with a relatively large capacity, has been that an unwanted spurt of fluid is expressed from the nozzle of the dispenser when the plunger reaches the end of an intake or return stroke.

This expression of fluid, caused by a pressure wave travelling though the fluid resident in the barrel when the plunger reaches the limit of its travel, has resulted in wastage of the fluid and, in medical applications, to the potential for incorrect dosages to be applied. This may be wasteful and/or dangerous.

In an attempt to mitigate the problem some dispensers of the prior art have used a length of soft hosing provided between the barrel and a container in which the fluid is stored, to attempt to reduce the magnitude of the pressure wave through expansion of the tube.

A problem with this method is that soft hose may react adversely with some chemicals due to the higher proportion of plasticiser in its chemical makeup. In general it may be desirable to use harder plastics as these may be more resistant to chemical attack.

The reference to any prior art in this specification is not, and should not be taken as, an acknowledgment or any form of suggestion that that prior art forms part of the common general knowledge in the field of endeavour to which the invention relates.

### OBJECT OF THE INVENTION

It is an object of a preferred embodiment of the invention to provide a dispensing apparatus which will overcome or ameliorate problems with such apparatus at present, or at least one which will provide the public with a useful choice.

Other objects of the present invention may become apparent from the following description, which is given by way of example only.

### SUMMARY OF THE INVENTION

According to one aspect of the present invention, there is provided a dispensing apparatus including:

a barrel;

a plunger moveable between a first position and a second position within said barrel, a head of said plunger and an inner wall of said barrel together defining a chamber of variable volume wherein said volume is at a maximum when said plunger is in said first position and said volume is at a minimum when said plunger is in said second position;

at least one fluid inlet valve means operable to allow fluid to flow into said chamber when said volume increases;

at least one fluid outlet valve means operable to allow fluid to flow out of said chamber when said volume decreases;

biasing means to bias said plunger towards said first position;

plunger actuating means operable to actuate said plunger towards said second position; and

plunger damping means to decelerate said plunger when said plunger is moving towards said first position before said plunger reaches said first position.

Preferably, said plunger damping means may include a resilient element provided between said plunger and a body of said dispensing apparatus.

Preferably, said plunger damping means may include a resilient element provided between said head of said plunger and said body of said dispensing apparatus.

Preferably, said damping means may include a spring member.

Preferably, said damping means may include a substantially U shaped spring member.

Preferably, said damping means may be connectable to said head of said plunger.

Preferably, at least one of said first position and said second position may be adjustable.

According to a second aspect of the present invention, there is provided a an animal health applicator including:

a barrel;

a plunger moveable between a first position and a second position within said barrel, a head of said plunger and an inner wall of said barrel together defining a chamber of variable volume wherein said volume is at a maximum when said plunger is in said first position and said volume is at a minimum when said plunger is in said second position;

at least one fluid inlet valve means operable to allow fluid to flow into said chamber when said volume increases;

at least one fluid outlet valve means operable to allow fluid to flow out of said chamber when said volume decreases;

biasing means to bias said plunger towards said first position;

plunger actuating means operable to actuate said plunger towards said second position; and

plunger damping means to decelerate said plunger when said plunger is moving towards said first position before said plunger reaches said first position.

According to a third aspect of the present invention there is provided an animal health applicator including:

a barrel;

a plunger moveable between a first position and a second position within said barrel, a head of said plunger and an inner wall of said barrel together defining a chamber of variable volume wherein said volume is at a maximum when said plunger is in said first position and said volume is at a minimum when said plunger is in said second position;

at least one fluid inlet valve means operable to allow fluid to flow into said chamber when said volume increases;

at least one fluid outlet valve means operable to allow fluid to flow out of said chamber when said volume decreases;

biasing means to bias said plunger towards said first position;

plunger actuating means operable to actuate said plunger towards said second position; and

a substantially U shape spring member connected to said head of said plunger to decelerate said plunger when said plunger is moving towards said first position before said plunger reaches said first position.

According to a still further aspect of the present invention, a dispensing apparatus is substantially as herein described with reference to the accompanying drawings.

Further aspects of the invention, which should be considered in all its novel aspects, will become apparent from the following description given by way of example of possible embodiments of the invention.

### BRIEF DESCRIPTION OF THE DRAWINGS

FIG. 1: Shows a cross sectional side view of a dispensing apparatus according to one possible embodiment of the present invention.

FIG. 2A: Shows a perspective view of the dispensing apparatus of FIG. 1 with the barrel removed for clarity and the plunger in the second position.

FIG. 2B: Shows a perspective view of the dispensing apparatus of FIG. 1 with the barrel removed for clarity, the plunger in the first position, and the resilient element deformed against the dosage control part.

### DESCRIPTION OF PREFERRED EMBODIMENTS OF THE INVENTION

The term “fluid” is used herein to include any liquid medium, whether pure liquid, mixtures of liquids, suspensions and the like.

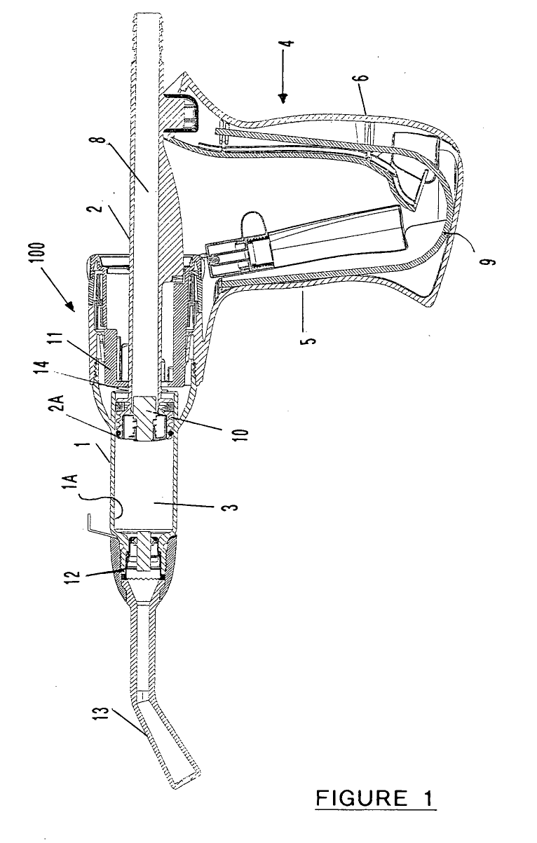

Referring first to FIG. 1, a dispensing apparatus according to one possible embodiment of the present invention is generally referenced 100.

The dispensing apparatus 100, in this embodiment configured as an animal health applicator, includes a barrel 1 within which is provided a plunger 2. The inner wall 1A of the barrel 1 and the plunger head 2a define a variable volume chamber 3.

The plunger 2 is able to reciprocate with the barrel 1 under the action of an actuating means generally referenced 4. The actuating means 4 includes first handle means 5 connectable to the barrel 1 and second handle means 6 connectable to the plunger 2. A conduit 8 may run from the head 2a to the opposite end of the plunger 2.

In use, the first and second handle means 5, 6 may be urged together to move the plunger 2 between a first position, in which the volume in the chamber 3 is at a maximum, towards a second position, in which the volume is at a minimum. A biasing means, in this case an elongate resilient spring member 9, provided between the first and second handle means 5, 6 may bias the first and second handle means 5, 6 apart and thereby bias the plunger 2 towards the first position, the movement of the plunger 2 in this direction hereinafter referred to as the intake or return stroke.

Those skilled in the art will appreciate that the first and second positions referred to above refer to the maximum and minimum volume of the chamber 3 for a given setting or configuration of the dispenser 100, and may be variable by an operator. Those skilled in the art will further appreciate that any suitable alternative biasing means may replace or supplement member 9.

In the embodiment illustrated the second position is defined by the setting of a dosage control part 11 which limits the stroke of the plunger 2. The configuration and working of the dosage control part is more fully explained in the applicant's co-pending New Zealand patent specification number 521084, and forms no part of the present invention. In alternative embodiments (not shown) the second position may be defined by, for example, the plunger head 2A abutting an end of the barrel 1.

The plunger 2 may be provided with an inlet valve means 10 which allows a fluid, for example a medicine, to pass from a fluid supply (not shown) via the conduit 8 into the chamber 3 when the volume or capacity of the chamber 3 increases, that is, when the plunger 2 moves towards the first position. In an alternative embodiment (not shown) the inlet valve means 10 may be provided in the barrel 1.

The barrel 1 may be provided with an outlet valve 12 which allows fluid to pass from the chamber 3 out of the barrel 1 when the volume of the chamber 3 decreases, that is, when the plunger 2 moves towards the second position.

The fluid may pass from the outlet valve 12 to an outlet conduit which may, for example, be a nozzle 13. In alternative embodiments the outlet conduit may be a needle or any other suitable outlet conduit.

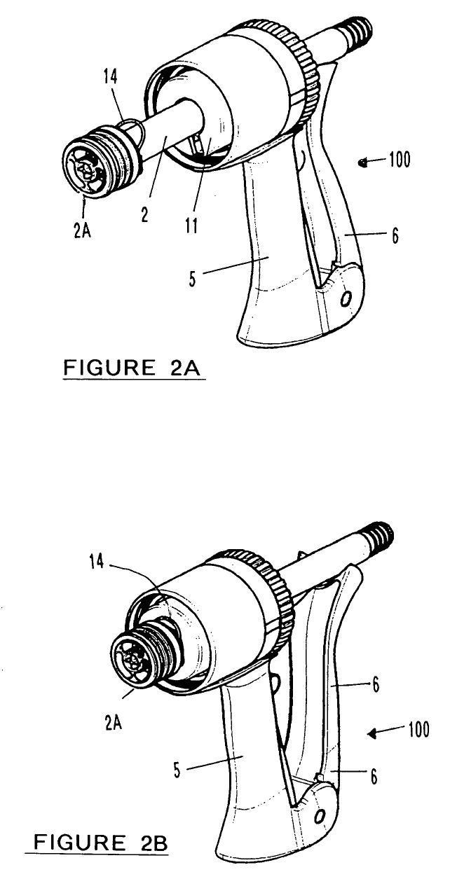

Referring next to FIGS. 2A and 2B, the plunger 2 may be provided with a damping means.

In the embodiment illustrated the damping means is a substantially U shape resilient member 14 which is connectable to the plunger 2 and is angled away from the plunger head 2A so that it abuts the dosage control part 11 before the plunger 2 reaches the first position as defined above.

The U shape resilient member 14 is made from a suitably resilient, substantially non-corroding material, for example light gauge stainless steel, which allows it to resiliently deform when it abuts the dosage control part 11, thereby decelerating the plunger 2 and providing a cushioning effect. The decelerating or cushioning effect of the resilient member 14 on the plunger 2 may reduce or even substantially eliminate the pressure wave which propagates through the fluid in the barrel 1 of the dispensers of the prior art when the plunger 2 reaches the end of its return stroke, that is, when the plunger 2 reaches the first position.

The U shape resilient member 14 is only one of the possible damping means contemplated by the applicant. In alternative embodiments (all not shown) a coil spring or a resilient concertinaed or bellows shape member may be provided around the plunger 2 between the plunger head 2A and the dosage control part 11, or any other suitable part of the dispenser body. In another embodiment a resilient polymeric member, such as an O-ring for example, may be provided on the back of the plunger head 2A and/or on the dosage control part 11, or may float on the plunger 2 between the plunger head 2A and the dosage control part 11.

As mentioned above, those skilled in the art will appreciate that some dispensers may be configured so that the first position is not defined by the plunger head 2A abutting a dosage control part 11. In some embodiments (all not shown) the first position may be defined by the plunger head 2A abutting another part of the body of the dispenser, or an end of the barrel 1. In alternative embodiments the first position may be defined by a projection on the plunger abutting a stop provided on a main body of the applicator. In yet further embodiments the first position may be defined by a projection on the plunger means abutting an adjustable dosage part.

In each of the examples listed above, the damping means may either be provided between the plunger and the element which limits the movement of the plunger, or may be separated from those parts. The damping means in all these examples begins to be deformed, and thus the plunger decelerated or cushioned, prior to the first position being reached.

Those skilled in the art will appreciate that the present invention provides a dispensing apparatus in which the tendency to express fluid when the plunger reaches the end of its intake stroke may be reduced over dispensing apparatus of the prior art.

While the dispensing apparatus described above is configured as an animal health applicator, those skilled in the art will appreciate that the dispensing apparatus may be configured for alternative uses such as, for example, food preparation.

Where in the foregoing description, reference has been made to specific components or integers of the invention having known equivalents, then such equivalents are herein incorporated as if individually set forth.

Although this invention has been described by way of example and with reference to possible embodiments thereof, it is to be understood that modifications or improvements may be made thereto without departing from the spirit or scope of the appended claims.

We claim:

1. A dispensing apparatus including:
a barrel;
a plunger moveable between a first position and a second position within said barrel, a head of said plunger and an inner wall of said barrel together defining a chamber of variable volume wherein said volume is at a maximum when said plunger is in said first position and said volume is at a minimum when said plunger is in said second position;
at least one fluid inlet valve means operable to allow fluid to flow into said chamber when said volume increases;
at least one fluid outlet valve means operable to allow fluid to flow out of said chamber when said volume decreases;
biasing means to bias said plunger towards said first position;
plunger actuating means operable to actuate said plunger towards said second position; and
plunger damping means to decelerate said plunger when said plunger is moving towards said first position before said plunger reaches said first position.

2. The dispensing apparatus of claim 1 wherein said plunger damping means includes a resilient element provided between said plunger and a body of said dispensing apparatus.

3. The dispensing apparatus of claim 2, wherein said plunger damping means includes a resilient element provided between said head of said plunger and said body of said dispensing apparatus.

4. The dispensing apparatus of claim 3 wherein said resilient element includes a substantially U shaped spring member.

5. The dispensing apparatus of claim 1, wherein said damping means includes a spring member.

6. The dispensing apparatus of claim 1 wherein said damping means is connectable to said plunger head.

7. The dispensing apparatus of claim 1 wherein at least one of said first position and said second position is adjustable.

8. An animal health applicator including:
a barrel;
a plunger moveable between a first position and a second position within said barrel, a head of said plunger and an inner wall of said barrel together defining a chamber of variable volume wherein said volume is at a maximum when said plunger is in said first position and said volume is at a minimum when said plunger is in said second position;
at least one fluid inlet valve means operable to allow fluid to flow into said chamber when said volume increases;
at least one fluid outlet valve means operable to allow fluid to flow out of said chamber when said volume decreases;
biasing means to bias said plunger towards said first position;
plunger actuating means operable to actuate said plunger towards said second position; and
plunger damping means to decelerate said plunger when said plunger is moving towards said first position before said plunger reaches said first position.

9. An animal health applicator including:
a barrel;
a plunger moveable between a first position and a second position within said barrel, a head of said plunger and an inner wall of said barrel together defining a chamber of variable volume wherein said volume is at a maximum when said plunger is in said first position and said volume is at a minimum when said plunger is in said second position;
at least one fluid inlet valve means operable to allow fluid to flow into said chamber when said volume increases;
at least one fluid outlet valve means operable to allow fluid to flow out of said chamber when said volume decreases;
biasing means to bias said plunger towards said first position;
plunger actuating means operable to actuate said plunger towards said second position; and
a substantially U shape spring member connected to said head of said plunger to decelerate said plunger when said plunger is moving towards said first position before said plunger reaches said first position.
